(19) United States
(12) Patent Application Publication
(10) Pub. No.: US 2006/0264803 A1
(43) Pub. Date: Nov. 23, 2006

(54) Suture collar

(75) Inventors: Chun Kee Lui, Monroeville, PA (US); Louis B. Goode, Cranberry Twp., PA (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Vascular Incorporated, Leechburg, PA (US)

(21) Appl. No.: 11/410,383

(22) Filed: Apr. 25, 2006

Related U.S. Application Data
(60) Provisional application No. 60/674,832, filed on Apr. 26, 2005.

(51) Int. Cl. A61N 1/30

(52) U.S. Cl. 604/19

(57) ABSTRACT

A collar for anchoring a lead in a vein. The collar includes a body member having axial ends and a throughbore extending between the axial ends. The throughbore is sized for receiving at least a portion of the lead. The body member includes one or more grooves disposed along its outer surface for receiving a suture for anchoring the lead. At least a portion of the inner surface of the body member includes structure sized and positioned to project into the throughbore for generating a tangential compression against the lead, thereby inhibiting slippage of the lead when the lead is received in the throughbore.

### RELATED APPLICATION

The present patent document claims the benefit of the filing date under 35 U.S.C. §119(e) of Provisional U.S. Patent Application Ser. No. 60/674,832, filed Apr. 26, 2005, which is hereby incorporated by reference.

### BACKGROUND

1. Technical Field

This invention relates to a suture collar for use in medical applications. More particularly, the invention relates to a suture collar that is receivable over a body of a cardiac lead, and is useful for minimizing undesired migration or slippage of the cardiac lead following implantation of the lead into a patient.

2. Background Information

In the course of treatment of certain cardiac conditions, such as a heart rhythm abnormality, a physician may elect to implant a medical device, such as a pacemaker or a defibrillator, into a pocket of the chest cavity of the patient. Alternatively, other devices, such as externally-positioned defibrillators, that are not implanted in the patient, but that perform many of the same functions as the implanted devices may also be utilized in the treatment of cardiac abnormalities. Cardiac devices of this type are normally provided to establish and/or maintain a desired beating rate, or “rhythm” of the patient's heart. In most cases, the devices are provided to prevent the heart from beating too slowly for proper functioning.

When such cardiac devices are utilized, a tip electrode is generally inserted through a vein into the atrium or ventricle of the heart. A lead is provided that extends between the tip electrode and the device, to establish an electrical connection between the device and the tip electrode. The lead commonly includes a coiled structure such as an electrical wire coil for conducting electrical signals (such as stimulating and/or sensing signals) between the cardiac device and the heart. Some leads include one or more coaxial or lateral helical wire coils having a lumen that extends the entire length of the wire coil or coils. Other leads may be made with a cable or a tightly wound coil without a lumen. In either case, the cable or wire coils are surrounded by an electrically insulating material such as a flexible tube, sheath or coating. The insulating material, generally formed of silicone or polyurethane, serves simultaneously to protect the cable and wire coils from body fluids, and in the case of bi-polar leads, to insulate the wire coils from one another.

When the device is a pacemaker, the pacemaker is implanted in a pocket in the chest cavity, and the lead is implanted such that it extends between the tip electrode and the pacemaker to complete the electrical connection. In this instance, the lead is inserted into a vein, such as a cephalic vein or the right external jugular vein. One end of the lead extends to the tip electrode that is fixed in place in an atrium or ventricle of the heart, and the other end extends to the implanted pacemaker unit.

Once the components of the device have been implanted, it is desired to minimize, if not eliminate altogether, migration within the patient's body of any of the components of the device, such as the lead. Migration of the pacemaker lead can lead to undesirable results, such as the severing of the electrical connection between the tip electrode and the pacemaker, and/or the drift of the lead to an undesirable site in the body.

Several techniques have been devised in an attempt to anchor the lead at the desired placement in the body. One common technique involves the use of one or more sutures or a similar connection mechanism to attach the lead to a vessel in which the lead has been implanted, or to the tissue surrounding the lead. Typically, a suture collar is provided which is movable along the lead to a location where it is desired to secure the lead to the vessel or to the underlying tissue. The suture collar may be provided with one or more spaced circumferential grooves that are sized to receive a suture that is passed around the vessel or through the underlying tissue, and tied within the groove. The use of sutures and other prior art connection mechanisms is discussed in, for example, U.S. Pat. No. 4,516,584, titled “Suture Collar”, U.S. Pat. No. 4,683,895, titled “Suture Sleeve Anchoring Device”, U.S. Pat. No. 4,553,961, titled “Suture Sleeve with Structure for Enhancing Pacing Lead Gripping”, and U.S. Pat. No. 5,746,711, titled “Suture Sleeve with Circumferential Lead Locking Device.” Each of the afore-mentioned patents is incorporated herein by reference.

The arrangements described in the recited patents do not always result in a secure anchor for the lead. As a result, notwithstanding the preventative efforts, the lead may still slip through the collar. If the lead slips through a collar, either the electrode tip or the pacemaker device may become displaced from the lead, thereby destroying the electrical connection between the pacemaker and the electrode tip, and rendering the pacemaker useless. In addition, in some instances a collar that has been tied with a suture may result in the exertion of a concentrated force on the lead. Such force may result in partial or total severing of the lead insulation, and possibly even of the lead itself. Severing of the lead insulation reduces the ability of the insulation to protect the cable and wire coils from body fluids. Severing of the lead itself causes the electrical connection between the pacemaker and the electrode tip to be broken.

It is desired to provide a suture collar that overcomes the problems of prior art. It is further desired to provide a suture collar that is easy to use, inexpensive to make, inhibits slippage of the lead through the collar, and that protects the lead from the concentrated forces exerted by the sutures.

### BRIEF SUMMARY

The problems of the prior art are addressed by the present invention. In one form thereof, the invention comprises a collar for anchoring a lead in a vein in which the lead has been implanted. The collar comprises a body member having axial ends and a throughbore extending between the axial ends. The body member has an outer surface and an inner surface, wherein the inner surface defines the throughbore. The throughbore is sized for receiving at least a portion of the lead therein. The body member includes one or more grooves disposed along its outer surface, wherein the grooves are sized for receiving a suture for use in anchoring the lead. At least a portion of the inner surface includes structure sized and positioned for inhibiting slippage of the lead when the lead is received in the throughbore.

In another form thereof, the present invention comprises a method for forming a suture collar for use in inhibiting slippage of a lead implanted in a vein of a patient. A collar body is provided having an outer surface and an inner surface. The collar body has open axial ends, and a throughbore defined by the inner surface that extends between the open ends. Irregularities are formed on at least a portion of the inner surface, which irregularities extend radially inwardly into the throughbore and are positioned for generating a tangential compression against the lead. The irregularities may comprise a grit material that is applied to the inner surface.

In yet another form thereof, the present invention comprises an articulating collar assembly for anchoring a lead in a vein in which the lead has been implanted. The articulating collar assembly includes a plurality of body members, and a generally flexible joinder member disposed between adjacent body members. Each of the body members has axial ends and a throughbore extending between the axial ends. The body members further have an outer surface and an inner surface, wherein the inner surface defines the throughbore. The throughbore is sized for receiving at least a portion of the lead therein.

### BRIEF DESCRIPTION OF THE DRAWINGS

FIG. 1 is a side view of a suture collar according to an embodiment of the present invention;

FIG. 2 is a sectional view of the suture collar of FIG. 1;

FIG. 3 is an end view of the suture collar of FIG. 1;

FIG. 4 is a sectional view of another embodiment of the suture collar as shown in FIG. 2

FIG. 5 is a view of the suture collar of FIG. 1, wherein a pin is provided for holding the suture collar in an open position;

FIG. 6 is a sectional view of another embodiment of a suture collar according to the present invention, including internal gripping rings;

FIG. 7 is a sectional view of another embodiment of a suture collar according to the present invention, including internal gripping elements;

FIG. 8 is a sectional view of another embodiment of a suture collar according to the present invention, including internal helices;

FIG. 9 is a sectional view of another embodiment of a suture collar according to the present invention, including internal perforations; and

FIG. 10 is a side view of another embodiment of a suture collar.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the inventive suture collar, as well as the axial ends of various related components. The term “proximal” is used in its conventional sense to refer to the end of the suture collar (or component thereof) that is closest to the operator during use of the collar. The term “distal” is used in its conventional sense to refer to the end of the suture collar (or component thereof) that is initially inserted into the patient, or that is closest to the patient.

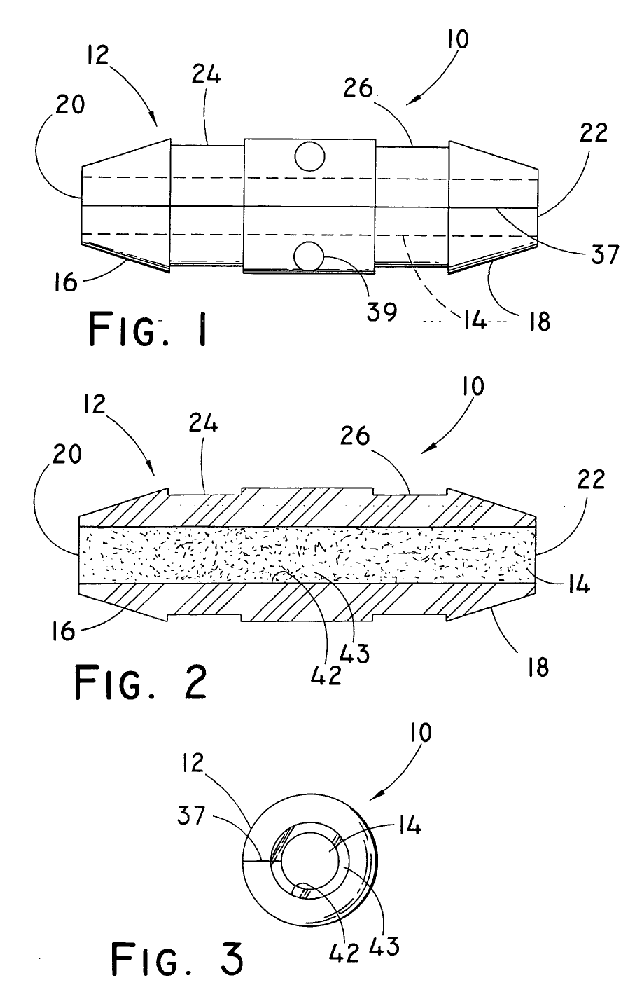

Referring now to the drawings, a preferred embodiment of an inventive suture collar is shown in FIGS. 1-3. FIG. 1 illustrates a side view of suture collar 10. FIG. 2 illustrates a sectional view of collar 10 of FIG. 1, and FIG. 3 illustrates an end view of collar 10. Suture collar 10 is utilized for anchoring a lead, such as a cardiac lead, at a desired location in the body following implantation of the lead. The lead may extend from a cardiac device, such as a pacemaker or defibrillator, in well-known fashion and pass through an incision in a vein, such as the subclavian vein, to the patient's heart. Suture collar 10 is generally positioned on the lead at a location near the incision, and is secured to the vein or adjacent tissue to prevent dislodgement of the lead.

A cardiac lead, as the term is used herein, refers to a lead that is used in connection with a heart-related device. Non-limiting examples of cardiac leads that may be anchored by the inventive device include pacemaker leads, defibrillator leads, coronary sinus leads, and left ventricular pacing leads. Although primarily discussed herein in connection with cardiac leads, the invention is not so limited. Rather, the inventive collar may also be used to anchor other types of leads, such as neurological pacing and stimulation leads. In addition to anchoring the various leads previously described, the inventive collar may also be used to anchor other types of medical devices, such as catheters, to a designated site in the body of the patient. Although the discussion hereinafter generally refers to use of the device to anchor a cardiac lead, those skilled in the art will appreciate that the teachings of this invention are applicable to numerous other uses, each of which is considered within the scope of the invention.

As stated, it is known in the medical arts to utilize a suture collar to anchor a medical lead, such as a cardiac lead. Such collars are generally used to anchor a lead in place, while at the same time protecting the lead from the concentrated force that would otherwise be exerted directly on the lead by the suture. Although such leads have been widely used, a recurring problem involves slippage of the lead through the collar. Such slippage may result in the severing of the electrical connection between the tip electrode and the pacemaker, and/or the drift of the lead to an undesirable site in the body. The inventive suture collar addresses this problem of undesired slippage.

In the preferred embodiment shown in FIGS. 1-3, suture collar 10 comprises an elongated generally cylindrical main body 12. Main body 12 defines a central elongated bore 14 extending longitudinally therethrough from an opening 20 in a first axial end to an opening 22 in a second axial end. Tapered end portions 16, 18 may be provided at opposing axial ends of main body 12. In the embodiment shown, grooves 24, 26 are formed in main body 12 by any conventional means. Grooves 24, 26 are sized and positioned to receive sutures in well-known fashion. Those skilled in the art will appreciate that main body 12 can be structured in numerous different ways to provide one or more grooves, conduits, recesses, receptacles or like structure for receiving the sutures. For convenience, the term “grooves” is used herein in an inclusive sense to refer to any such structures. Although FIGS. 1 and 2 illustrate the presence of two grooves, any number of grooves may be employed in a particular case.

In prior art suture collars, the inner surface portion of the collar that receives and surrounds the elongated bore is substantially cylindrical in shape and has a generally smooth, continuous surface. In addition, the inner surface is sized to slidably receive the cardiac lead therethrough. Similarly, in most prior art collars, the collar is fabricated in a manner such that its main body is homogeneous throughout. Thus, the composition of the portion of the collar that contacts the lead is the same as the composition of the remainder of the lead.

The inventive collar differs from such prior art collars by not having a generally smooth inner surface, or in other words, by having an irregular surface, and/or by not being formed from a homogeneous composition. In the embodiment of FIGS. 1-3, collar body 12 is fabricated such that an inner surface of the bore is not homogeneous with the remainder of collar body 12. As a further alternative, selected portions of inner surface 42 can even be structured to be non-homogeneous with other portions of inner surface 42. As a result, when inner surface 42 is compressed against a lead, inner surface 42 contacts or otherwise deforms in an irregular manner against the surface of the lead. This irregular deformation generates tangential anchoring or compression against the lead. In addition, tangential friction may be established to resist slippage between the collar and the lead.

A suture collar body 12 may be molded or otherwise fabricated from a generally flexible material, such as an elastomer. Silicone is a preferred material for this purpose, although other elastomers known to be suitable for medical applications, such as polyurethanes and expanded polytetrafluoroethylene (ePTFE), may be substituted.

In the embodiment of FIGS. 1-3, irregularities are formed on inner surface 42 of main body 12. In a preferred embodiment, the irregularities are formed by coating the inner surface with a gritty material 43. The gritty material 43 may be fabricated from a material such as aluminum oxide or other biocompatible ceramics, or from stainless steel and other biocompatible metals and metal alloys. The gritty material provides the inner surface with a roughened, irregular surface. This surface generates compression against the lead in tangential fashion, thereby resisting slippage of the lead from within the collar bore.

Gritty material 43 may be applied to inner surface 42 of suture collar 10 by any convenient manner. Preferably, inner surface 42 is initially coated with a suitable adhesive. The coating may be applied by any conventional method, such as by direct application with a manual applicator, or by spraying the adhesive onto the inner bore surface. The particular adhesive selected for use will be capable of firmly adhering to the material of collar main body 12. Thus, for example, when silicone is used as the main body composition, it is preferred to use a silicone adhesive. Silicone adhesives are well known for their ability to firmly adhere to a structure formed of silicone. Similarly, when the sleeve is formed from other compositions, a suitable adhesive capable of firmly adhering to the particular composition should be utilized. Those of ordinary skill in the art can readily select a suitable adhesive for use with a particular main body material.

Following application of the adhesive, collar bore 14 is filled with the gritty material 43. As a result, particles of gritty material 43 adhere to the adhesive-coated inner surface 42 of collar main body 12 in random fashion. Excess gritty material is then poured out of the inner bore, and the adhesive is allowed to cure. Those skilled in the art can readily devise a suitable combination of curing time and temperature for a particular sleeve composition and adhesive to promote satisfactory curing in a particular application.

Although the gritty material was applied to collar inner surface 42 in generally random fashion in the embodiment shown in FIG. 2, this is not necessarily required, and other arrangements may be substituted. For example, in the alternative embodiment shown in FIG. 4, the gritty material 43 is applied in a manner to comprise one or more annular rings along inner surface 42. With this arrangement, the adhesive would, of course, be applied in a manner consistent with the desired pattern of the gritty material. Instead of rings, other formations or patterns, such as helices, lines, dots, etc., may be substituted. This arrangement may provide enhanced tangential gripping at certain locations of the lead. As a still further alternative, portions of inner surface 42 can be coated with gritty material in random fashion as in FIG. 2, and other portions may be coated to comprise a specific pattern, such as the pattern of rings in FIG. 4.

Although the gritty material is separately applied to the inner surface of the suture collar in the embodiments described above, this need not be the case. Rather, in an alternative embodiment, the gritty material can be mixed with the material of the collar body material prior to molding or otherwise fabricating the collar. In this manner, the separate application step can be eliminated. However, in this case the gritty material will generally be randomly distributed throughout the entire collar body, rather than solely on the inner surface.

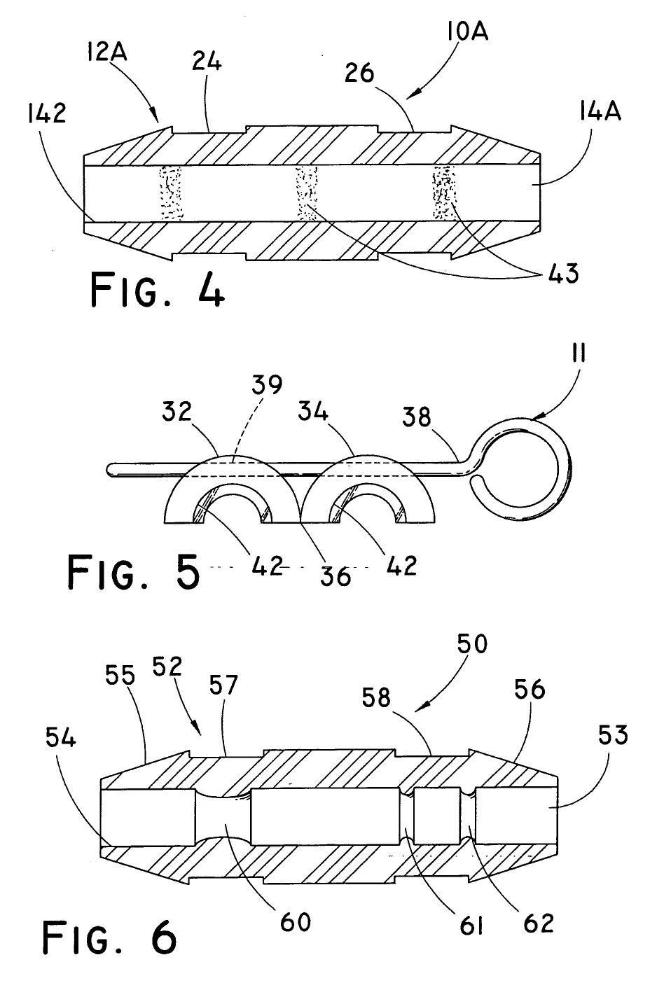

Following application of the layer of gritty material, collar 10 is preferably slit and pinned into an open position, as shown in FIG. 5. Axial slit 37 may be formed in a wall of collar main body 12 by any conventional means, such as by cutting with a razor or other straightedge. As shown in FIG. 5, suture collar 10 may then be folded open to comprise two collar segments, or “halves” 32, 34, which segments are at least connected along one axial end 36. Prior to use, collar halves 32, 34 are preferably maintained in the “open” position shown in FIG. 5 by any suitable mechanism, such as pin 38. In the embodiment shown, pin 38 is inserted through a bore 39 that extends through a portion of collar body 12 to thereby maintain collar halves 32, 34 in the open position shown. Those of ordinary skill in the art will appreciate that maintaining collar halves 32, 34 in the open position by utilizing the pin and bore arrangement shown in FIG. 5 represents only one of many possible mechanisms that could be utilized to maintain the open configuration, each of which is considered within the scope of the invention. Preferably, slit 37 is only cut through one side wall of the collar as shown in FIG. 3, although if desired, the slit may also extend through a portion of the opposing side wall.

Although the collar 10 as illustrated in FIG. 5 comprises body halves 32, 34, this arrangement is exemplary only, and those skilled in the art will appreciate that main body 12 can be formed to have more than two body segments. Alternatively, it is not necessary to form the collar body to have body segments as described. Rather, the collar can simply be maintained in its generally cylindrical configuration, and slipped over the lead in conventional fashion. However, due to the gritty or irregular nature of the inside surface of collar 10, it is believed that the better practice is to engage the collar with the selected portion of the lead by positioning the two collar halves around the lead portion, and then closing the collar around the lead. Threading the lead through the gritty or irregular inner surface of the collar may result in dislodgement of gritty particles from the inner collar surface. When the two collar halves are properly positioned around the lead, little if any dislodgement is expected. The two collar halves are then maintained in the closed position around the lead by the force of the suture acting on the collar.

Collar 10 may be provided with a myriad of different combinations of dimensions. Appropriate dimensions for a particular collar depend upon several factors, such as the size and type of the lead to be anchored, and the type of suture or other anchoring mechanism to be applied for the intended use. In one preferred embodiment, collar 10 may have a length of about 0.775 inch (2 cm), and a maximum width of about 0.23 inch (0.6 cm) at tapered portions 16, 18, and a maximum width of about 0.206 inch (0.5 cm) at grooves 24, 26. Those skilled in the art will appreciate that these, and other, dimensions provided herein are only examples of possible dimensions, and those skilled in the art can readily select an appropriate set of dimensions for a particular application. A preferred effective diameter of the inner passageway (e.g., the passageway diameter following application of the gritty material) will approximate the diameter of the lead. In this manner, the lead will receive favorable circumferential gripping, ideally along 360 degrees of its circumferential surface, and the sleeve would begin to grip onto the lead after only minimal tightening of the sutures onto the suture collar.

Leads are typically supplied in combination with suture sleeves. As a result, proper size matching of a lead with a suitable sleeve will generally not be problematic. When they are not supplied in combination, a certain amount of trial and error may be required to provide a sleeve that properly fits over a particular lead. Any such experimentation is considered routine, and it is well within the capability of one skilled in the art to properly match and sleeve with a lead.

Another way to fabricate a suture collar to obtain the benefits of the present invention is to modify the collar inner surface 42 to alter the properties across its surface. For example, various structures, such as rings, dots, helices, stripes, and the like, can be incorporated onto inner surface 42 such that they extend radially inwardly from inner body surface. In this manner, the structures promote the irregularity and discontinuity of the surface, and provide a gripping surface that is capable of gripping the body of the lead. Such structures can be formed of the same material used to form collar body 12, or from a different material.

One example of such a structure is shown in FIG. 6. FIG. 6 is a sectional view of suture collar 50. Suture collar 50 comprises an elongated generally cylindrical main body 52, a central elongated bore 53, an inner body surface 54, tapered end portions 55, 56, and grooves 57, 58, as before. In this embodiment, annular rings 60, 61, 62 are provided along inner body surface 54. Although the embodiment of FIG. 6 shows three rings, this is exemplary only. The collar may be provided with any number of rings, wherein each individual ring may have any one of an endless number of possible thicknesses and cross-sections. The rings may also have various geometries, such as rectangular, triangular and curved.

Other non-limiting examples of collars having modifying structures are illustrated in FIGS. 7-9. FIG. 7 illustrates a collar 70 having main body 72, central elongated bore 73, an inner body surface 74, tapered end portions 75, 76, and grooves 77, 78, as before. Internal protrusions of various shapes and sizes may project radially inwardly from inner surface 74 into elongated bore 73. The protrusions can comprise one or more of an endless number of geometrical configurations, such as cylindrical, hemispherical, rectangular, truncated cone, trapezoidal, etc. Non-limiting examples of many such configurations are illustrated in the figure.

In the embodiment of FIG. 8, collar 90 comprises main body 92, central elongated bore 93, an inner body surface 94, tapered end portions 95, 96, and grooves 97, 98, as before. In this case, one or more internal helices 99 are provided along inner surface 94. Helices 99 need not necessarily have the same diameters, and need not project a common distance into bore 93. The helices may have various geometries, such as rectangular, triangular and curved.

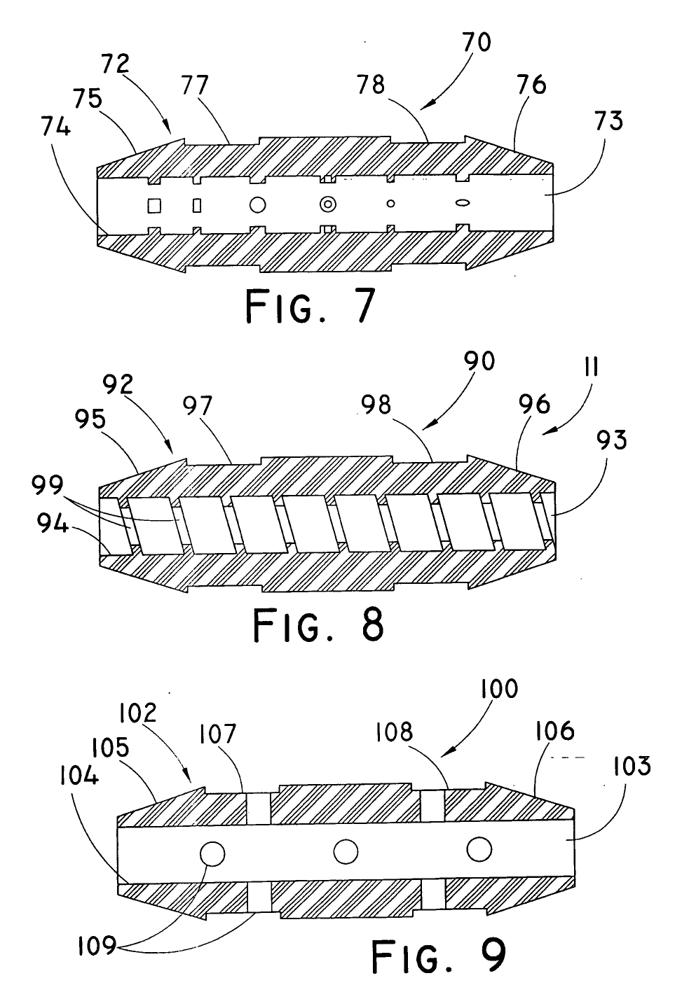

In the embodiment of FIG. 9, collar 100 comprises main body 102, central elongated bore 103, an inner body surface 104, tapered end portions 105, 106, and grooves 107, 108, as before. In this embodiment, one or more perforations 109 extend through body 102. The perforations need not have the same shape and/or diameter, and may be randomly distributed along main body 102.

Another alternative comprises the application of a coating to a specific portion of the inner bore, and not to other portions, thereby enhancing the non-homogeneity, or irregularity, of the inner bore. This coating can be applied to the specific inner bore portion in a manner similar to that described above with reference to the adhesive coating, or in any other manner. The coating may be provided such that the coated portions of the surface of the inner bore differ in thickness from adjoining non-coated, or lesser coated, portions. As yet another alternative, the coating may be applied in a manner such that coated portions of the inner bore differ in hardness, or durometer, from adjoining non-coated, or lesser coated, portions.

As still another alternative, the properties of at least a portion of the inner bore surface can be altered by, e.g., chemical or physical means. Such means are applied in a manner such that the shape, feel, and/or texture of portions of the inner surface is altered in a manner to further promote the irregularity, on non-homogeneity, of the internal passageway. Non-limiting examples of such conversion means include chemically treating or etching designated portions of the inner surface, and/or thermally treating portions of the inner surface by a suitable mechanism (such as a laser) to convert the texture of selected portions of the inner body surface from, e.g., a soft texture to a hard texture.

Thus, a collar formed according to the present invention may have all, or some, of the features described in the various embodiments described hereinabove. Those skilled in the art will appreciate that a goal of this structure is to provide a non-homogeneous, or irregular, inner surface to the collar body, by any one or more of a multitude of possible means. As a result, portions of the irregular inner collar surface can grip the outer surface of the lead to inhibit slippage of the lead.

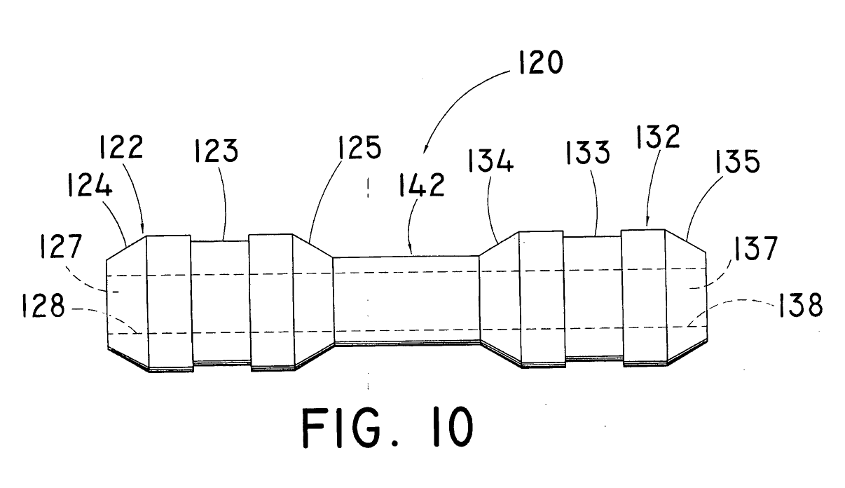

Another embodiment of the present invention comprises an articulating suture collar 120, as illustrated in FIG. 10. In the embodiment shown, suture collar 120 includes dual collar bodies 122, 132, joined by generally flexible middle portion 142. Collar bodies 122, 132, include respective grooves 123, 133, and respective tapered portions 124, 134 and 125, 135. Each one of bodies 122, 132 includes an elongated bore 127, 137 defined by an inner surface 128, 138. Flexible middle portion 142 enables collar 120 to be applied to a non-linear lead, or in other words, to a lead that is positioned in a manner such that the lead has a curved orientation. Similarly, collar 120 may be utilized when it is to be sutured or otherwise attached to a non-linear (e.g., curved) structure in the body.

The presence of the flexible middle portion allows the device to conform to a multitude of different orientations. Body portions 122, 132 are retained in the body in conventional manner, such as by sutures that are received in grooves 123, 133. Although the embodiment shown has two collar bodies and one articulating section joining the bodies, collar 120 can be provided with as many bodies, and flexible articulating sections, as desired for a particular use. Similarly, the bodies to be joined can have any configuration suitable for such use, and are not limited by the particular configuration shown in the figure. As still another alternative, a structure can be provided with a plurality of successive collar bodies and articulating sections, whereupon a physician may simply trim off the number of collar bodies and articulating sections as may be desired for a particular use. If desired, respective inner surfaces 128, 138 of collar 120 may also be provided with any of the gripping features described previously, and illustrated in the figures, or with no gripping features.

Collar bodies 122, 132 may be formed of the same or similar material, and are preferably formed from any of the collar materials previously recited. Flexible middle portion 142 may be formed from any material having sufficient flexibility to bend as required, and sufficient strength to maintain joinder of body portions 122, 132. Typically, middle portion may be formed of the same materials utilized to form collar bodies 122, 132. Non-limiting examples of suitable materials include silicone, polyurethane and ePTFE. Those skilled in the art will appreciate that collar 120 may conveniently be molded or otherwise fabricated as a single, integral structure.

It is therefore intended that the foregoing detailed description be regarded as illustrative rather than limiting, and that it be understood that it is the following claims, including all equivalents, that are intended to define the spirit and scope of this invention.

1. A collar for anchoring a lead in a vein in which the lead has been implanted, comprising:
a body member having axial ends and a throughbore extending between said axial ends, said body member having an outer surface and an inner surface, said inner surface defining said throughbore, said throughbore sized for receiving at least a portion of said lead therein, said body member including one or more grooves disposed along said outer surface, said one or more grooves sized for receiving a suture for use in anchoring said lead, at least a portion of said inner surface including structure sized and positioned for inhibiting slippage of said lead when said lead is received in said throughbore.

2. The collar of claim 1, wherein said structure for inhibiting slippage comprises a roughened portion of said inner surface.

3. The collar of claim 2, wherein said roughened portion comprises a grit material affixed to said inner surface.

4. The collar of claim 3, wherein said grit material comprises aluminum oxide grits.

5. The collar of claim 3, wherein said grit material is affixed to a designated portion of said inner surface, and wherein another designated portion of said inner surface does not have a grit material affixed thereto.

6. The collar of claim 1, wherein said structure extends radially inwardly into said throughbore from said inner surface.

7. The collar of claim 6, wherein said structure comprises at least one of rings, dots, stripes and helices.

8. The collar of claim 1, wherein said structure comprises a coating over said portion of said inner surface, said coating extending radially inwardly into said throughbore.

9. The collar of claim 8, wherein said coating has a higher durometer than a durometer of a non-coated portion of said inner surface.

10. The collar of claim 1, wherein said structure comprises a portion of said inner surface modified by at least one of chemical and physical treatment.

11. The collar of claim 10, wherein said structure comprises a chemically etched portion of said inner surface.

12. The collar of claim 10, wherein said portion is physically treated to alter the durometer of said treated portion relative to other portions of said body member.

13. The collar of claim 12, wherein said treatment comprises exposing said portion to laser energy.

14. The collar of claim 1, wherein said body member comprises a member selected from the group consisting of silicone, polyurethane, ePTFE, and mixtures thereof.

15. The collar of claim 14, wherein said body member includes a grit material incorporated therein for inhibiting said slippage.

16. A method for forming a suture collar for use in inhibiting slippage of a lead implanted in a vein of a patient, comprising:
providing a collar body, said body having an outer surface and an inner surface, said collar body further having open axial ends and a throughbore defined by said inner surface extending between said open axial ends; and
forming irregularities on at least a portion of said inner surface, said irregularities extending radially inwardly into said throughbore and positioned for generating a tangential compression against said lead.

17. The method of claim 16, wherein said step of forming irregularities comprises:
applying an adhesive to at least a portion of said inner surface;
inserting a grit material into said throughbore, such that at least a portion of said grit material adheres to said inner surface; and
removing a non-adhered portion of said grit material from said throughbore.

18. The method of claim 17, further comprising the steps of:
forming an axial slit through a wall of said collar body along a length of said collar body;
opening said collar body along said slit to define two body halves, each of said body halves including opposing diametric portions of said throughbore; and
providing a mechanism to selectively maintain said body halves in an open position.

19. An articulating collar assembly for anchoring a lead in a vein in which the lead has been implanted, comprising:
a plurality of body members, each body member having axial ends and a throughbore extending between said axial ends, each body member further having an outer surface and an inner surface, said inner surface defining said throughbore, said throughbore sized for receiving at least a portion of said lead therein, said body member including one or more receptacles disposed along said outer surface for receiving a suture for use in anchoring said lead; and
a generally flexible joinder member disposed between adjacent body members.

20. The articulating collar assembly of claim 19, wherein at least some of said body members include radially-inwardly directed structure from said inner surface, said structure sized and positioned for inhibiting slippage of said lead when said lead is received in said throughbore.
